(19) United States
(12) Patent Application Publication
(10) Pub. No.: US 2021/0307372 A1
(43) Pub. Date: Oct. 7, 2021

(54) METHOD OF PRODUCING A BEVERAGE FROM GREEN PEPPER OR RED HOT PEPPER JUICE AND POWDER

(71) Applicant: Kieu Hoang, Westlake Village, CA (US)

(72) Inventor: Kieu Hoang, Westlake Village, CA (US)

(21) Appl. No.: 15/278,127

(22) Filed: Sep. 28, 2016

Related U.S. Application Data
(60) Provisional application No. 62/233,903, filed on Sep. 28, 2015.

(51) Int. Cl. A23L 33/185; A61K 36/81; A61K 38/16; A61K 9/00; A23L 2/66; A23L 33/105; A23L 33/00

(52) U.S. Cl. CPC A23L 33/185; A61K 36/81; A61K 38/168; A23V 2002/00; A23L 2/66; A23L 33/105; A23L 33/40; A61K 9/0056

(57) ABSTRACT

A food composition for human consumption comprising a plurality of pepper ingredients, wherein the plurality of pepper ingredients comprise ground flesh, seed, stem, and skin from green peppers or red hot peppers, and wherein the plurality of pepper ingredients further comprise one or more KH Pepper proteins. A method of treating a certain disease in a patient comprising administering the food composition to a patient in need thereof, wherein the food composition has a concentration of KH Pepper protein above 0%.

### RELATED APPLICATIONS

The present patent application claims priority to provisional U.S. Patent Application No. 62/233,903 filed Sep. 28, 2015, which was filed by the inventor hereof and is incorporated by reference herein in its entirety.

### TECHNICAL FIELD

The present subject matter relates to a process for producing beverages on the basis of juice and powder from the green pepper or red hot pepper and further relates to a process for obtaining fresh juice and powder suitable for human consumption from the green pepper or red hot pepper, wherein the complete pepper is grinded and used to make juice and powder. The present subject matter provides a process for obtaining juice and powder from the green pepper or red hot pepper, wherein the skin, seed, flesh, and stem of the pepper are all ground and centrifuged to make juice and powder.

### BACKGROUND

Since 7500 BC or earlier, chili peppers have been a part of the human diet. Though there are only a few commonly used species, there are many cultivars and methods of preparing chili peppers that have different names for culinary use. Green and red bell peppers, for example, are the same cultivar of C. annuum, immature peppers being green. In the same species are the jalapeno, the poblano (referred to as ancho when dried), New Mexico (also known as chile Colorado), Serrano, and other cultivars.

Peppers are commonly broken down into three groups: bell peppers, sweet peppers, and hot peppers. Most popular pepper varieties are seen as falling into one of these categories, or as a cross between them. Chili peppers have several purposes, including for culinary, psychological, medicinal, and crop defense uses, etc. Capsaicin, which gives hot peppers their intensity when ingested or applied topically, also is considered and used as a safe and effective topical analgesic agent in the management of arthritis pain, herpes zoster-related pain, diabetic neuropathy, mastectomy pain, and headaches.

A 2015 cohort study in China found that eating foods containing chili peppers at least twice a week led to a 10 percent reduced mortality rate. Furthermore, with all else being equal, when eating foods containing chili peppers 6 to 7 days a week, there was a 14 percent relative risk reduction in total mortality. It was found that there was an inverse correlation between eating fresh chilies and diabetes, which inverse correlation was not found in the remainder of the cohort.

### SUMMARY

In an embodiment, the present subject relates to a food composition for human consumption comprising a plurality of pepper ingredients, wherein the plurality of pepper ingredients comprises ground flesh, seed, stem, and skin from green peppers or red hot peppers, and wherein the plurality of pepper ingredients further comprise one or more KH Pepper proteins.

In this regard, the present subject matter further relates to a method of treating certain diseases in a patient comprising administering a food composition for human consumption to a patient in need thereof, the composition comprising a plurality of pepper ingredients, wherein the plurality of pepper ingredients comprise ground flesh, seed, stem, and skin from green peppers or red hot peppers, wherein the plurality of pepper ingredients further comprise one or more KH Pepper proteins such that the composition has a concentration of KH pepper proteins above 0%.

In one embodiment in this regard, KH Pepper healthy cells from the KH Pepper proteins, after administration to the patient, send signals to damaged or sick cells, thereby triggering synthesis of proteins to transform the damaged or sick cells to become healthy, wherein the KH Pepper healthy cells send signals to other undamaged cells to synthesize proteins to protect the other undamaged cells from damage, infection, and from being prone to DNA and other cellular alterations, and wherein the KH Pepper healthy cells send signals to the patient's body to produce new cells that are healthy, thereby preventing the new cells from being affected by intracellular and extracellular damaging signals.

### BRIEF DESCRIPTION OF THE DRAWINGS

FIG. 1 shows an image of the SDS-Page of green pepper, green pepper whole grinded suspension, and red hot pepper.

FIG. 2 shows an image of the base peaks of the samples.

FIG. 3 shows an image of the SDS-Page of green pepper fractionation supernatant.

FIG. 4 shows an image of the SDS-Page of green pepper fractionation paste.

FIG. 5 shows an image of the SDS-Page of red hot pepper fractionation supernatant.

FIG. 6 shows an image of the SDS-Page of red hot pepper fractionation paste.

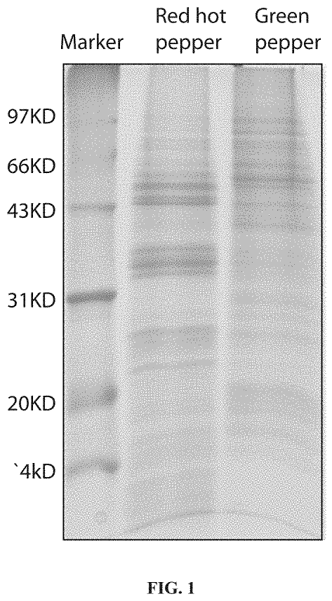

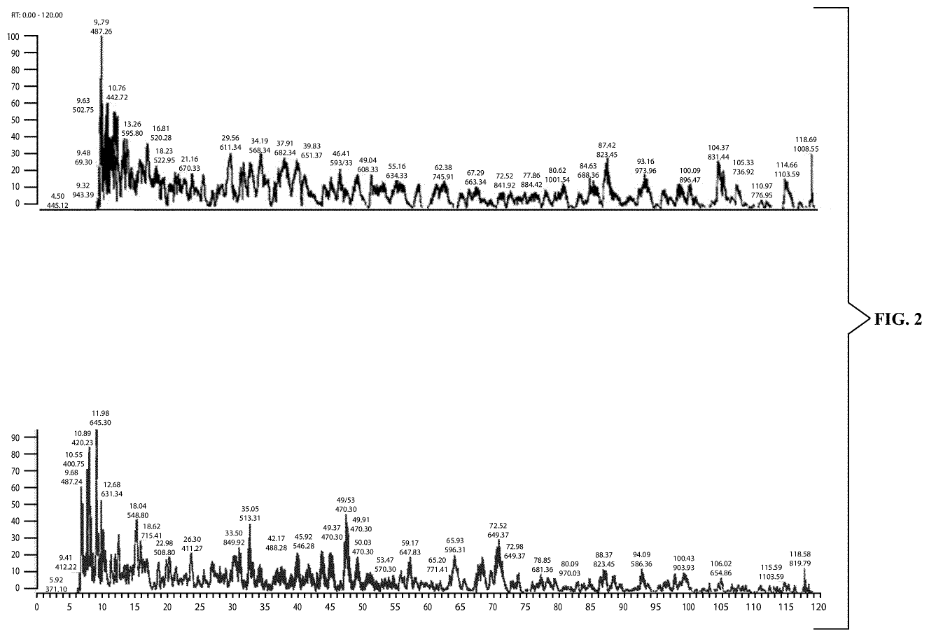

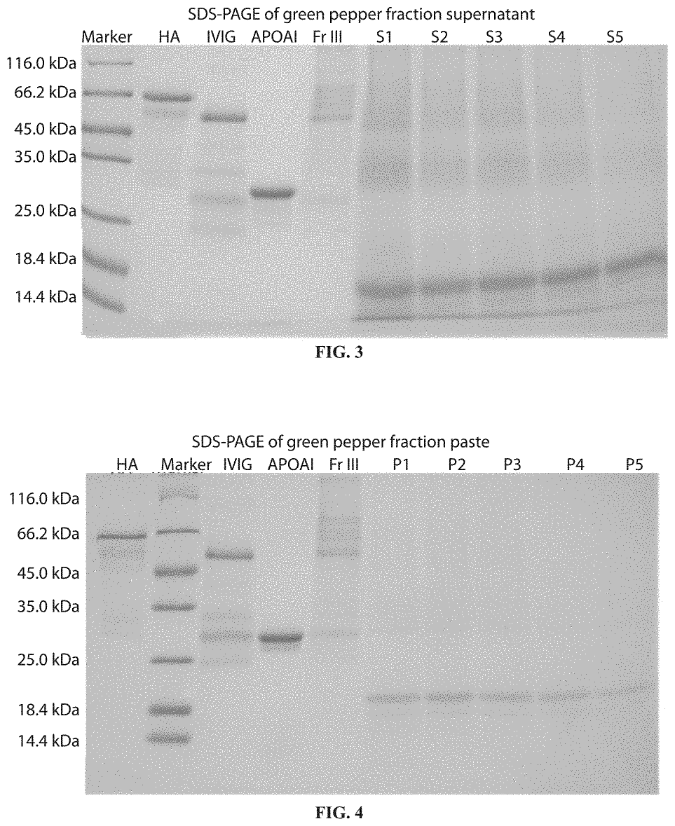

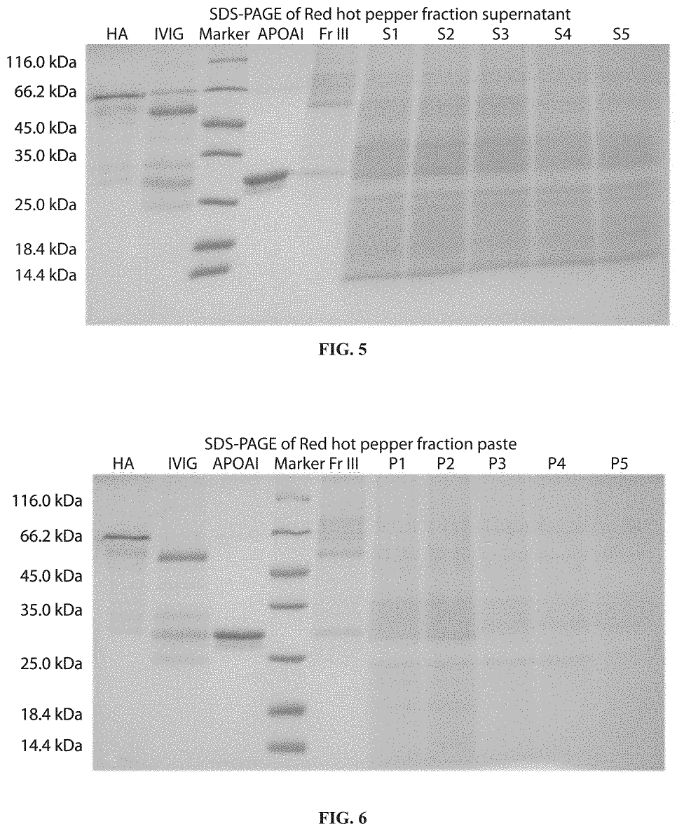

### DETAILED DESCRIPTION

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

Throughout the application, descriptions of various embodiments use “comprising” language; however, it will be understood by one of skill in the art, that in some specific instances, an embodiment can alternatively be described using the language “consisting essentially of or” consisting of”.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about”. Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

An embodiment of the present subject matter is directed to a food composition for human consumption comprising a plurality of pepper ingredients, wherein the plurality of pepper ingredients comprise ground flesh, seed, stem, and skin from green peppers and/or red hot peppers, and wherein the plurality of pepper ingredients further comprise one or more KH Pepper proteins. The present subject matter provides a process for obtaining juice and powder from green pepper and/or red hot pepper, wherein the skin, seed, flesh and stem are all grinded and centrifuged to make juice and powder.

There are a total of 1349 proteins found in green pepper. Among them, 777 are putative newly found KH good healthy proteins synthesized by KH good healthy cells. There are a total of 1032 proteins found in red hot pepper. Among them, 597 are putative newly found KH good healthy proteins synthesized by KH good healthy cells.

In an embodiment, the present subject matter describes a method for the production of juice and powder from the green peppers or red hot peppers. The green pepper or red hot pepper is treated immediately after harvest. The fresh green pepper or red hot pepper is washed in a cleaning bath at about 0-100° C. The whole green pepper or red hot pepper is smashed in a grinder to cut the thick stem at about 0-100° C. The raw materials are collected and ground in a superfine mill at about 0-100° C. The raw juice is collected. The pH of the raw juice is checked and should be about 1-14. The raw juice is centrifuged at low to high rpm with a normal centrifuge at about −30-30° C. The precipitate and supernatant are collected.

Water at two to four times the volume is added to the precipitate and mixed about 10-100 minutes and centrifuged again at low to high rpm with normal centrifuge at about −30-60° C. The precipitate and supernatant are collected.

Water at one to two times the volume is added, then mixed about 10-100 minutes and centrifuged again at low to high rpm with normal centrifuge at about −30-60° C. The precipitate and supernatant are collected. All the supernatant is mixed for 10-100 minutes at about 0-100° C. The pH of the supernatant is checked and should be about 2.5-10. The supernatant juice is centrifuged at moderate to high rpm with a disc centrifuge at about −30-60° C., and the juice is collected. The juice pH is checked and should be about 2.5-10.

In an embodiment, to make juice product, the collected juice is homogenized at 40-60 MPa. The juice is sterilized at 130−140° C. for 5-20 seconds with a pipe sterilizer. The clear juice is bottled at 25−35° C. The pH of the juice is checked.

In an embodiment, to make a powder product, the collected juice is concentrated in a single-effect falling film evaporator at 65−85° C. until the Brix value is 30-50 degrees. The concentrated juice is transferred to the centrifugal spray dryer to produce powder. All of the five precipitates are mixed and capsulized.

Another aspect of the present subject matter relates to a food composition for human consumption comprising a plurality of pepper ingredients, wherein the plurality of pepper ingredients comprise ground flesh, seed, stem, and skin from green peppers or red hot peppers, and wherein the plurality of pepper ingredients further comprise one or more KH Pepper proteins.

In an embodiment, the food composition of the present subject matter is a powder, juice or other beverage, food supplement, or concentrate. In an embodiment, the food composition of the present subject matter is a beverage and the KH Pepper protein comprises at least one of the 777 newly found KH proteins from green peppers or at least one of the 597 newly found KH proteins from red hot peppers. In an embodiment, the food composition of the present subject matter is a juice comprising KH healthy cells to synthesize KH proteins. In an embodiment, the food composition of the present subject matter is a mixed powder comprising KH healthy cells to synthesize KH proteins. In an embodiment, the food composition of the present subject matter further comprises living cells.

In an embodiment, the KH Pepper protein of the present subject matter comprises proteins having a molecular weight similar to molecular weights of human immunoglobulin, APOA-1, human albumin ApoA1 proteins, and heavy chains and light chains of immunoglobulins.

An embodiment of the present subject matter relates to a method of treating a certain disease in a patient comprising administering the food composition of the present subject matter to a patient in need thereof, wherein the food composition has a concentration of KH Pepper protein above 0%. In an embodiment, the certain disease is cancer and the administration inhibits growth of cancer cells.

In an embodiment of the present subject matter, healthy cells, formed after administration of the KH Pepper proteins, signal to damaged or sick cells, thereby triggering synthesis of proteins by RNA to transform the damaged or sick cells to become healthy, wherein the healthy cells send signals to other undamaged cells to synthesize proteins to protect the other undamaged cells from damage, infection, and from being prone to DNA and other cellular alterations, and wherein the healthy cells send signals to the patient's body to produce new cells that are healthy, thereby preventing the new cells from being affected by intracellular and extracellular damaging signals. In an embodiment the KH Pepper proteins comprise two or more combined newly found KH proteins.

In an embodiment, the juice is a mixed beverage that will contain a certain number of KH Good Healthy cells which synthesize KH Good healthy proteins. In an embodiment, the powder is a mixed food supplement that will also contain a certain number of KH Good Healthy cells which synthesize KH Good healthy proteins.

In an embodiment, green pepper or red hot pepper have KH GOOD HEALTHY CELLS in which the RNA synthesizes good and Healthy KH proteins that send signals to the DAMAGED, SICK, AND BAD CELLS that trigger synthesis of good proteins that transform these cells to become GOOD healthy cells; send signals to the other currently undamaged cells to trigger synthesis of good proteins to protect them from being DAMAGED, INFECTED and PRONE to DNA and other cellular alterations; and send signals to the body to produce new cells that are healthy and forbid them from being affected by intra- and extracellular damaging signals.

In an embodiment, the green pepper or red hot pepper contains proteins whose molecular weights are similar to the molecular weight of the human proteins of immunoglobulin and APOA-1 and other larger or smaller molecular weights of other human proteins, especially human albumin ApoA1 and both heavy chains and light chains of immunoglobulins. In an embodiment, living cells are found in the dried powder and beverages.

In an embodiment, any or two or more of combined newly found proteins send signals to the DAMAGED, SICK, AND BAD CELLS that trigger synthesis of good proteins that transform these cells to become GOOD healthy cells; send signals to the other currently undamaged cells to trigger synthesis of good proteins to protect them from being DAMAGED, INFECTED and PRONE to DNA and other cellular alterations; and send signals to the body to produce new cells that are healthy and forbid them from being affected by intra- and extracellular damaging signals.

In an embodiment, any two or more combined newly found proteins can be used to prevent, treat, and/or cure any kind of diseases, cancers, ailments, and deficiencies in a human patient, such as solid tumor and blood cancers.

### EXAMPLES

The objective of this study was to quantify Cholesterol/Cholesteryl Ester (TC), HDL Cholesterol (HDLC), LDL/VLDL Cholesterol (LDLC/VLDLC), and Triglyceride (TG) concentration in RAAS products. Tests were performed by one of the top ten CRO labs in the world. The highest level of HDL among tested items was found in KIEU HOANG™ wine, where red wine KH110 contains the highest level of HDL with 0.180/uL. The red hot pepper, KH 132, has the highest triglyceride (2.928 mmol/L) of all products tested.

The Cholesterol/Cholesteryl Ester Quantitation Kit provides a simple method for sensitive quantification of free cholesterol, cholesteryl esters, or both by colorimetric or fluorometric methods. The majority of the cholesterol in blood is in the form of cholesteryl esters which can be hydrolyzed to cholesterol by cholesterol esterase. Cholesterol is then oxidized by cholesterol oxidase to yield H₂O₂ which reacts with a sensitive cholesterol probe to produce color (λmax=570 nm) and fluorescence (Ex/Em=535/590 nm). The assay detects total cholesterol (cholesterol and cholesteryl esters) in the presence of cholesterol esterase or free cholesterol in the absence of cholesterol esterase in the reaction.

BioVision's HDL and LDL/VLDL Cholesterol Quantification Kit provides a simple quantification method of HDL and LDL/VLDL after a convenient separation of HDL from LDL and VLDL (very low-density lipoprotein) in serum samples. In the assay, cholesterol oxidase specifically recognizes free cholesterol and produces products which react with probe to generate color (λ=570 nm) and fluorescence (Ex/Em=538/587 nm). Cholesterol esterase hydrolizes cholesteryl ester into free cholesterol, therefore, cholesterol ester and free cholesterol can be detected separately in the presence and absence of cholesterol esterase in the reactions.

Likewise, the Triglyceride Quantification Kit provides a sensitive, easy assay to measure triglyceride concentration in variety of samples. In the assay, triglycerides are converted to free fatty acids and glycerol. The glycerol is then oxidized to generate a product which reacts with the probe to generate colorimetric (spectrophotometry at λ=570 nm) and fluorometric (Ex/Em=535/590 nm) methods. The kit can detect 1 pmol-10 nmol (or 1˜10000 μM range) of triglyceride in various samples listed below in Table 1:

TABLE 1

| | TC | HDL | LDL/VLDL | TG |
|---|---|---|---|---|
| Name | (μg/μl) | (μg/μl) | (μg/μl) | (mmol/L) |
| KH 101 | 0.000 | 0.006 | 0.001 | 0.013 |
| KH 102 | 0.002 | 0.004 | 0.001 | 0.094 |
| KH 103 | 0.005 | 0.007 | 0.004 | 0.018 |
| KH 104 | 0.091 | 0.052 | 0.003 | 0.051 |
| KH 105 | 0.049 | 0.046 | 0.002 | 0.064 |
| KH 106 | 0.029 | 0.028 | 0.001 | 0.021 |
| KH 107 | 0.001 | 0.003 | 0.001 | 0.017 |
| KH 108 | 0.000 | 0.002 | 0.001 | 0.014 |
| KH 109 | 0.025 | 0.030 | 0.004 | 0.207 |
| KH 110 | 0.134 | 0.180 | 0.037 | 1.684 |
| KH 111 | 0.015 | 0.009 | 0.010 | 1.865 |
| KH 112 | 0.000 | 0.006 | 0.003 | 0.017 |
| KH 113 | 0.001 | 0.012 | 0.001 | 0.021 |
| KH 114 | 0.002 | 0.008 | 0.002 | 0.232 |
| KH 115 | 0.005 | 0.054 | 0.003 | 0.053 |
| KH 116 | 0.001 | 0.012 | 0.001 | 0.027 |
| KH 117 | 0.000 | 0.010 | 0.001 | 0.066 |
| KH 118 | 0.008 | 0.031 | 0.004 | 0.102 |
| KH 119 | 0.004 | 0.035 | 0.002 | 0.011 |
| KH 120 | 0.000 | 0.021 | 0.003 | 0.031 |
| KH 121 | 0.000 | 0.007 | 0.002 | 0.019 |
| KH 122 | 0.000 | 0.008 | 0.001 | 0.003 |
| KH 123 | 0.002 | 0.016 | 0.003 | 0.104 |
| KH 124 | 0.012 | 0.024 | 0.002 | 0.015 |
| KH 125 | 0.000 | 0.002 | 0.001 | 0.002 |
| KH 126 | 0.000 | 0.002 | 0.001 | 0.001 |
| KH 127 | 0.002 | 0.014 | 0.002 | 0.113 |
| KH 128 | 0.003 | 0.006 | 0.001 | 0.014 |
| KH 129 | 0.001 | 0.005 | 0.001 | 0.006 |
| KH 130 | 0.004 | 0.054 | 0.002 | 0.015 |
| KH 131 | 0.013 | 0.014 | 0.001 | 0.031 |
| KH 132 | 0.005 | 0.007 | 0.002 | 2.928 |
| KH 133 | 0.013 | 0.012 | 0.002 | 0.029 |
| KH 134 | 0.003 | 0.005 | 0.002 | 0.054 |
| KH 201 | 0.002 | 0.002 | 0.003 | 0.027 |
| KH 202 | 0.000 | 0.003 | 0.002 | 0.017 |
| KH 203 | 0.000 | 0.002 | 0.002 | 0.003 |
| KH 204 | 0.000 | 0.003 | 0.001 | 0.187 |
| KH 205 | 0.000 | 0.002 | 0.001 | 0.006 |
| KH 206 | 0.000 | 0.002 | 0.001 | 0.007 |
| KH 207 | 0.000 | 0.003 | 0.003 | 0.023 |
| KH 208 | 0.002 | 0.002 | 0.002 | 0.046 |
| KH 209 | 0.003 | 0.004 | 0.002 | 0.007 |
| KH 210 | 0.063 | 0.003 | 0.034 | 0.752 |
| KH 211 | 0.000 | 0.002 | 0.001 | 0.002 |
| KH 212 | 0.000 | 0.002 | 0.002 | 0.012 |
| KH 213 | 0.000 | 0.002 | 0.002 | 0.015 |
| KH 214 | 0.004 | 0.003 | 0.002 | 0.118 |
| KH 215 | 0.003 | 0.004 | 0.003 | 0.258 |
| KH 216 | 0.003 | 0.006 | 0.003 | 0.318 |
| KH 217 | 0.003 | 0.006 | 0.003 | 0.223 |
| KH 301 | 0.002 | 0.018 | 0.003 | 0.079 |
| KH 302 | 0.000 | 0.005 | 0.002 | 0.285 |
| KH 303 | 0.000 | 0.005 | 0.002 | 0.264 |
| KH 304 | 0.036 | 0.014 | 0.007 | 0.301 |
| KH 305 | 0.034 | 0.015 | 0.007 | 0.302 |
| KH 306 | 0.036 | 0.014 | 0.009 | 0.297 |
| KH 307 | 0.037 | 0.016 | 0.008 | 0.296 |
| KH 308 | 0.039 | 0.015 | 0.008 | 0.289 |
| KH 309 | 0.001 | 0.004 | 0.002 | 0.120 |

With the information contained herein, various departures from precise descriptions of the present subject matter will be readily apparent to those skilled in the art to which the present subject matter pertains, without departing from the spirit and the scope of the below claims. The present subject matter is not considered limited in scope to the procedures, properties, or components defined, since the preferred embodiments and other descriptions are intended only to be illustrative of particular aspects of the presently provided subject matter. Indeed, various modifications of the described modes for carrying out the present subject matter which are obvious to those skilled in chemistry, biochemistry, or related fields are intended to be within the scope of the following claims.

I claim:

1. A food composition for human consumption comprising:
a plurality of pepper ingredients,
wherein the plurality of pepper ingredients comprise ground flesh, seed, stem, and skin from green peppers or red hot peppers, and
wherein the plurality of pepper ingredients further comprise one or more KH Pepper proteins.

2. The food composition of claim 1, wherein the food composition is a powder, juice or other beverage, food supplement, or concentrate.

3. The food composition of claim 2, wherein the food composition is a beverage and the KH Pepper protein comprises at least one of the 777 newly found KH proteins from green peppers or at least one of the 597 newly found KH proteins from red hot peppers.

4. The food composition of claim 2, wherein the food composition is a juice comprising KH healthy cells to synthesize KH proteins.

5. The food composition of claim 2, wherein the food composition is a mixed powder comprising KH healthy cells to synthesize KH proteins.

6. The food composition of claim 1, wherein the KH Pepper protein comprises proteins having a molecular weight similar to molecular weights of human immunoglobulin, APOA-1, human albumin ApoA1 proteins, and heavy chains and light chains of immunoglobulins.

7. The food composition of claim 2, further comprising living cells.

8. A method of treating a certain disease in a patient comprising administering the food composition of claim 1 to a patient in need thereof, wherein the food composition has a concentration of KH Pepper protein above 0%.

9. The method of claim 8, wherein the certain disease is cancer and the administration inhibits growth of cancer cells.

10. The method of claim 8, wherein healthy cells, formed after administration of the KH Pepper proteins, signal to damaged or sick cells, thereby triggering synthesis of proteins by RNA to transform the damaged or sick cells to become healthy,
wherein the healthy cells send signals to other undamaged cells to synthesize proteins to protect the other undamaged cells from damage, infection, and from being prone to DNA and other cellular alterations, and
wherein the healthy cells send signals to the patient's body to produce new cells that are healthy, thereby preventing the new cells from being affected by intracellular and extracellular damaging signals.

11. The method of claim 10, wherein the KH Pepper proteins comprise two or more combined newly found KH proteins.
